(19) United States
(12) Patent Application Publication
(10) Pub. No.: US 2025/0303052 A1
(43) Pub. Date: Oct. 2, 2025

(54) A Syringe for Processing Fat Grafts and Related Methods

(71) Applicant: Orchid Holdings LLC, Lexington, KY (US)

(72) Inventors: David Kirn, Lexington, KY (US); William Whitman, Lexington, KY (US)

(21) Appl. No.: 18/864,310

(22) PCT Filed: May 19, 2023
(86) PCT No.: PCT/US2023/022923

§ 371 Date: Nov. 8, 2024

Related U.S. Application Data
(60) Provisional application No. 63/344,314, filed on May 20, 2022.

(51) Int. Cl. A61M 1/00

(52) U.S. Cl. CPC A61M 1/893; A61M 2205/7545

(57) ABSTRACT

A syringe for withdrawing an aspirate from a body for micro fat grafting includes a barrel having a first barrel portion with an inner wall with a lesser diameter than an inner wall of a second portion. A filter for separating fat cells from the aspirate extends within the second portion of the barrel. An absorbent is positioned between the filter and the second portion inner wall for absorbing the aspirate passing through the filter. A plunger moves within the barrel along the first portion inner wall and the filter and includes a gasket forming a seal with the first portion inner wall and an inner wall of the filter. Proximal and distal supports engage the filter and the barrel. In another embodiment, the filter has flared ends for engaging the barrel. A method of obtaining fat cells for micro fat grafting using the syringe is provided.

This application claims the benefit of U.S. Provisional Patent Application No. 63/344,314, filed May 20, 2022, the disclosure of which is incorporated herein by reference.

### TECHNICAL FIELD

The invention generally relates to a syringe for harvesting, processing, and re-introducing a small volume fat graft into patients.

### BACKGROUND

The process of micro fat grafting involves the suction aspiration, e.g. liposuction and other known techniques, of small amounts of fat from a harvest site of a patient, and subsequent re-injection of the fat into a grafting site of the same patient. The aspirate is harvested from an area of the patient in which an excess of fat resides, such as the patient's thighs, abdomen, or hips. The aspirate typically includes fat cells, local anesthetic, oil from ruptured fat cells, and blood. In order to deliver a high-quality fat cell graft, viable fat cells must be gently separated from the other components of the aspirate. Excess or rough processing of the fat cells can damage the viable cells. If the fat cells are damaged, they can necrotize and die after re-introduction into the body leading to poor long-term results. Hence, the goal is to re-introduce or graft as many viable fat cells as possible and minimize the re-introduction of the non-viable cells and other components of the aspirate.

The most common micro fat grafting technique involves an initial liposuction of aspirate with a syringe and a harvesting cannula. The surgeon creates a vacuum inside the syringe which draws the aspirate through the cannula and into the barrel of the syringe. Although the aspirate could be directly re-injected into the grafting site, it is preferential to separate the fat cells from the remaining aspirate since only the fat cells are capable of maintaining viability in the grafting site. There are currently several methods for separating fat cells from the remaining aspirate for grafting including, processing the aspirate via centrifuge, filtering or washing the aspirate, using a washing vessel, and/or simply waiting for gravity to separate the fat cells from the remaining aspirate.

With most existing methods of fat cell harvesting, the fat cells must be removed from a harvesting container, processed, and transferred into one or more syringes for re-injection. Hence, these methods involve substantial handling of the fat cells and possibility of generating trauma thereto. Even more, significant mechanical forces are applied to the fat cells with centrifugation and some of the known filtration methods which can lead to cellular damage. Transferring and processing the fat cells also requires significant time and exposes the fat cells to potential contamination during the open transfer process. Accordingly, a need exists to effectively and efficiently harvest fat cells, separate the viable fat cells from the oil and fluid of the remaining aspirate in a gentle and timely manner, minimize or eliminate vessel transfer, i.e., utilize a closed system, and re-introduce viable fat cells back into the patient thereby minimizing the number of apoptotic cells in the graft.

In the following description, there are shown and described several preferred embodiments of a syringe for withdrawing an aspirate from a body for micro fat grafting and related methods of securing fat cells for micro fat grafting using the syringe. As it should be realized, the syringes and related methods are capable of other, different embodiments and their several details are capable of modification in various, obvious aspects all without departing from the assemblies as set forth and described in the following claims. Accordingly, the drawings and descriptions should be regarded as illustrative in nature and not as restrictive.

### SUMMARY OF THE INVENTION

In accordance with the purposes and benefits described herein, a syringe for withdrawing an aspirate from a body for micro fat grafting is provided. The syringe may be broadly described as including a barrel having first and second portions. The first barrel portion has an inner wall with a lesser diameter than an inner wall of the second portion. The syringe further includes a filter for separating the fat cells from the aspirate extends within the second portion of the barrel, an absorbent positioned between the filter and the second portion inner wall for absorbing the aspirate passing through the filter, and a plunger for longitudinal movement within the barrel along the first portion inner wall and the filter. The plunger includes a gasket attached to a proximal end of the plunger forming a seal with the first portion inner wall in a first position and an inner wall of the filter in a second position.

In accordance with another aspect of the invention, a syringe for withdrawing an aspirate from a body for micro fat grafting is provided. The syringe may be broadly described as including a barrel having first and second portions. The first barrel portion having an inner wall with a lesser diameter than an inner wall of the second portion. The syringe further includes a filter for separating the fat cells from the aspirate extends within the second portion of the barrel, an absorbent positioned between the filter and the second portion inner wall for absorbing the aspirate passing through the filter, and a plunger for longitudinal movement within the barrel along the first portion inner wall and the filter. In this embodiment, the plunger includes a gasket attached to a proximal end of the plunger forming a seal with the first portion inner wall in a first position. Even more, an inner diameter of the first portion inner wall and an inner diameter of an inner wall of the filter are substantially the same.

In another possible embodiment, the gasket sealingly engages the barrel first portion inner wall in a first position wherein the plunger is fully inserted into the barrel, a second position wherein the plunger is retracted to a distal end of the barrel first portion, and any position therebetween.

In yet another possible embodiment, the barrel second portion inner wall defines a notch for receiving a proximal end of the filter.

In one other possible embodiment, an inner diameter of the notch is substantially the same as a diameter of an outer wall of the proximal end of the filter.

In still another possible embodiment, the notch is positioned such that a distal end of the barrel first portion abuts the proximal end of the filter.

In yet still one other possible embodiment, the proximal end of the filter is outwardly flared.

In another possible embodiment, the syringe further includes a second gasket sealingly engaging the notch and the proximal end of the filter.

In one other possible embodiment, a distal end of the filter is outwardly flared.

In still another possible embodiment, the filter includes a proximal support.

In yet another possible embodiment, the proximal support receives the proximal end of the filter.

In another possible embodiment, an inner diameter of the notch is substantially the same as a diameter of an outer wall of the proximal support.

In still another possible embodiment, the notch is positioned such that a distal end of the barrel first portion abuts the proximal support.

In yet one more possible embodiment, the proximal support sealingly engages at least the notch.

In still yet another possible embodiment, the proximal support is at least one of a rubber, a polytetrafluoroethylene, a fluorosilicone, and a polyurethane.

In another possible embodiment, an outer wall of the filter and the barrel second portion inner wall at least partially define a chamber wherein the absorbent resides.

In still another possible embodiment, the syringe further includes a cap engaging a distal end of the barrel.

In one other possible embodiment, the cap further defines the chamber wherein the absorbent resides.

In yet another possible embodiment, the filter includes a distal support defining a central aperture through which the plunger extends.

In another possible embodiment, the distal support receives the distal end of the filter.

In one other possible embodiment, the distal support includes a disk defining the central aperture and having a radial outer edge.

In another possible embodiment, an inner diameter of the barrel second portion is substantially the same as an outer diameter of the radial outer edge of the distal support.

In yet another possible embodiment, a diameter of the central aperture is less than an inner diameter of the filter forming a proximal facing ledge such that a distal end of the filter abuts a face of the proximal facing ledge.

In still another possible embodiment, the distal support further includes an annular ring.

In yet still one other possible embodiment, the annular ring extends distally from the disk.

In one other possible embodiment, an inner diameter of the annular ring is larger than the diameter of the central aperture forming a distal facing ledge on the disk.

In another possible embodiment, a distal facing surface of the annular ring engages the cap to prevent longitudinal movement of the filter.

In still yet another possible embodiment, the annular ring is at least one of a rubber, a polytetrafluoroethylene, a fluorosilicone, and a polyurethane.

In yet one other possible embodiment, an inner side of the filter defines a second channel and an outer side of the filter and the barrel second portion inner wall at least partially define a chamber wherein the absorbent resides.

In still another possible embodiment, the second channel and the chamber are in continuous fluid communication when the plunger is retracted to any position between (1) a second position wherein the plunger is retracted to a distal end of the barrel first portion and (2) a third position wherein the plunger is retracted (a) to a distal end of the barrel second portion or (b) at the distal end of the barrel second portion.

In one other possible embodiment, the gasket contacts the filter inner wall around an entire circumference of the filter inner wall.

In another possible embodiment, the filter is a plastic or a metal. In yet another, the filter is a mesh.

In still another possible embodiment, the filter is secured in a cylindrical shape by one or more welds.

In yet another possible embodiment, the syringe further includes a second filter extending within the chamber and positioned between an outer wall of the filter and the absorbent.

In still yet another possible embodiment, the second filter is selected from the group consisting of a cotton gauze, a mesh filter, a fine mesh filter, a paper, or an absorptive paper.

In one other possible embodiment, the absorbent is a cotton. In yet another, the absorbent is a super absorbent-embedded cotton. In still yet another, the absorbent includes fibers.

In another possible embodiment, at least a first portion of the fibers wick and absorb oil. In another, at least a second portion of the fibers wick and absorb aqueous fluids.

In yet another possible embodiment, the absorbent includes a superabsorbent material. In still another, the superabsorbent material is a superabsorbent polymer.

In one other possible embodiment, the absorbent includes at least a first layer of fibers and a second layer of a superabsorbent material. In another, the absorbent includes fibers and a superabsorbent material homogeneously combined.

In yet still one other possible embodiment, at least a first portion of the fibers wick and absorb oil. In another possible embodiment, at least a second portion of the fibers wick and absorb aqueous fluids.

In another possible embodiment, the second filter and the absorbent form a cartridge.

In still another possible embodiment, the cartridge is generally cylindrical in shape and is positioned over the filter.

In yet one other possible embodiment, the syringe further includes at least one detent formed in the plunger for engaging a seal supported by the barrel to provide a tactile change in the plunger movement.

In still yet another possible embodiment, the seal is supported at a distal end of the barrel second portion preventing leaking along the plunger. In one other, the seal is supported by a distal support.

In another aspect of the invention, a method of obtaining fat cells from a body for micro fat grafting using a syringe includes the steps of retracting a plunger having a gasket attached to a proximal end thereof from a first position wherein the gasket engages an inner wall of a first portion of a barrel of the syringe in order to draw an aspirate from a harvest site of the body into the barrel first portion, further retracting the plunger to a second position exposing the aspirate to a filter and an absorbent for absorbing a portion of the aspirate, wherein the gasket engages an inner wall of the filter in the second position, and moving the plunger toward the first position such that an unabsorbed portion of the aspirate is no longer exposed to the absorbent.

In yet another possible embodiment, the inner wall of the first portion of the barrel has a lesser inner diameter than an inner diameter of a second portion of the barrel.

In another possible embodiment, the inner wall of the first portion of the barrel has an inner diameter substantially the same as an inner diameter of an inner wall of the filter.

In still yet another possible embodiment, the moving step includes the step of returning the unabsorbed portion of the aspirate to a grafting site of the body.

In one other possible embodiment, the method further includes the step of adding a wash solution to the aspirate drawn in the retracting step by further retracting the plunger from the first position to a first intermediate position.

In still another possible embodiment, the method further includes the step of adding a wash solution to the unabsorbed portion of the aspirate by again retracting the plunger from the first intermediate position to a second intermediate position.

In another possible embodiment, the method further includes the step of further retracting the plunger along the filter to the second position exposing the unabsorbed portion of the aspirate and wash solution mix to the filter and the absorbent.

In yet one other possible embodiment, the step of moving the plunger toward the first position occurs after at least one minute following retraction of the plunger to the second position.

In the following description, there are shown and described several preferred embodiments of the syringe for withdrawing an aspirate from a body for micro fat grafting and related methods of obtaining fat cells for micro fat grafting using the syringe. As it should be realized, the various syringes and methods are capable of other, different embodiments and their several details are capable of modification in various, obvious aspects all without departing from the syringes and methods as set forth and described in the following claims. Accordingly, the drawings and descriptions should be regarded as illustrative in nature and not as restrictive.

### BRIEF DESCRIPTION OF THE DRAWING FIGURES

The accompanying drawing figures incorporated herein and forming a part of the specification, illustrate several aspects of the syringes and methods and together with the description serve to explain certain principles thereof. In the drawing figures:

FIG. 1 is a perspective view of a syringe for withdrawing aspirate from a body for micro fat grafting with a partially transparent barrel;

FIG. 2 is a perspective view of the syringe with the barrel, absorbent, O-ring, and a portion of the filter assembly shown in section cut view;

FIG. 3 is a perspective view of a filter assembly;

FIG. 4 is a section cut partial end view of the syringe;

FIG. 5 is a partial section cut end view of an alternate embodiment of the filter assembly;

FIG. 6 is a partial section cut end view of another alternate embodiment of the filter assembly; and

FIG. 7 is a perspective view of an alternate filter assembly.

Reference will now be made in detail to embodiments of the syringe and related methods, examples of which are illustrated in the accompanying drawing figures, wherein like numerals are used to represent like elements.

### DETAILED DESCRIPTION

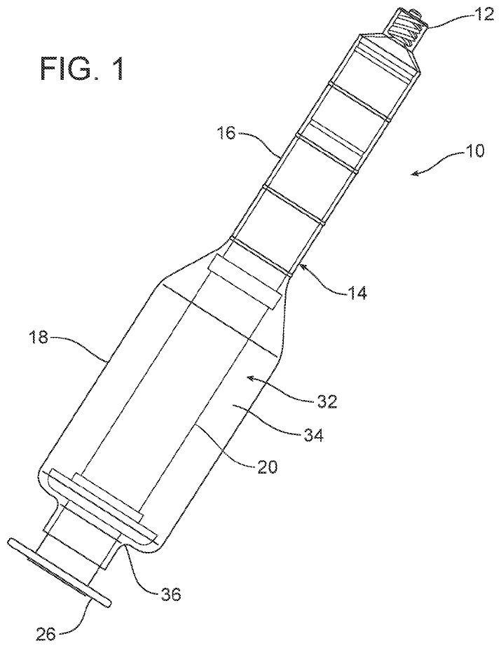

Reference is made to FIG. 1 which illustrates a syringe 10 for withdrawing an aspirate from a harvest site of a human body for micro fat grafting. The syringe 10 includes a fluid fitting 12 located at a proximal end of a barrel 14 consistent with an industry standard medical syringe. In the described embodiment, the fluid fitting 12 is a male Luer-Lok connection fitting designed to mate with a female Luer-Lok fitting. A fat harvesting cannula, hypodermic needle, liposuction cannula, or other extraction device (not shown) is connected to the fluid fitting 12 for use in withdrawing an aspirate.

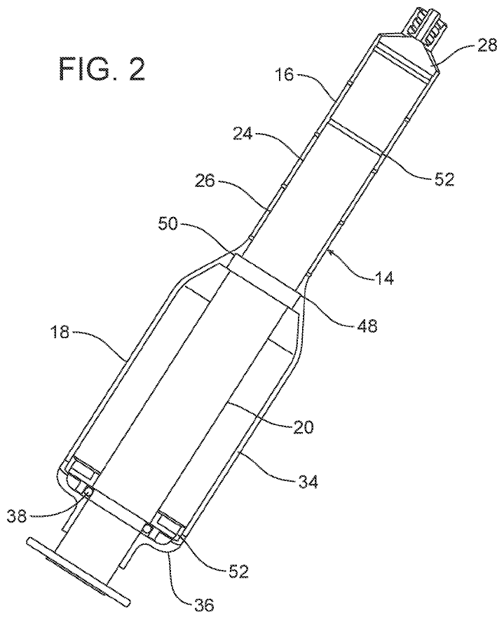

In the described embodiment, the barrel 14 includes first and second portions 16, 18. The first portion 16 has a lesser overall diameter than the second portion 18. As shown, a filter 20 extends longitudinally within the second portion 18 of the barrel 14. More specifically and as best shown in FIG. 2, a first channel generally defined by an inner wall 24 of the first portion 16 of the barrel 14 has an inner diameter substantially the same as an inner diameter of a second channel defined by the filter 20.

As further shown in FIG. 2, a plunger 26 moves longitudinally within the first and second channels defined by the inner wall 24 of the first portion 16 of the barrel 14 and the filter 20, and a gasket 28 attached to a proximal end of the plunger forms a seal against the inner wall and the filter. As is known in the art, retraction of the plunger 26 within one or both of the channels creates a vacuum allowing for the aspirate to be extracted from the body, through the extraction device, and into the channel(s). In other words, retraction of the plunger 26 from a first position draws the aspirate from the harvest site of the body into the barrel 14 of the syringe 10. In this embodiment, the plunger 26 is fully inserted in the syringe 10 in the first position and is retracted to a second position such that the gasket 28 is adjacent a distal end of the first portion of the barrel 16. In other embodiments, an external device or attachment may be utilized to withdraw and/or lock the plunger into a desired position.

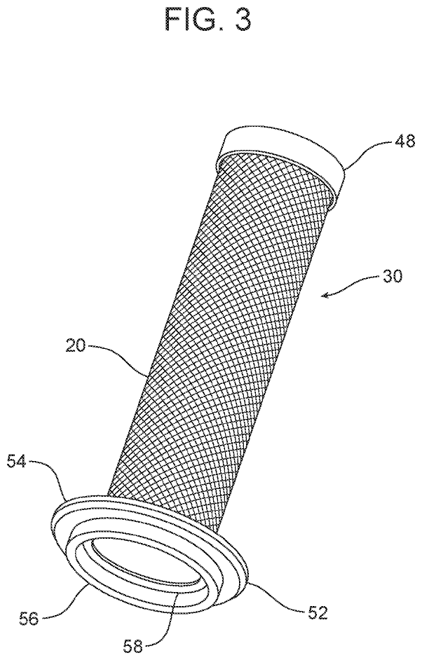

As shown in FIG. 3, the filter 20 is generally cylindrical in shape and forms a portion of a filter assembly 30. In the described embodiment, the filter 20 is an eighty mesh screen. The filter 20 may be a plastic polymer or metal and while the described embodiment utilizes an eighty mesh, a broader range of mesh may be utilized so long as the size of the openings are sufficient to limit, if not prevent, fat cells from traversing the filter so as to separate the fat cells from the remaining aspirate.

When fabricated into a cylindrical shape, the filter 20 provides adequate rigidity to define the aforementioned second channel and an interior wall of an absorptive chamber 32. Advantageously, utilization of the filter 20 minimizes a distance between the second channel within which the aspirate is drawn and an absorptive material 34 positioned within the chamber 32. Minimizing this distance significantly increases, if not maximizes, the absorptive effect on the aspirate over prior known devices that utilize sleeves and/or shutter devices.

Even more, the filter 20 eliminates gaps where aspirate can lodge between the channel and the absorptive material 34 thereby reducing the quality of the overall graft material. Such gaps may be created by additional structure designed to control absorption and/or support absorptive materials. Last, the apertures or fenestrations in the filter 20 are evenly distributed along an entirety of the filter allowing the absorptive material or absorbent 34 to be fully utilized. In other words, absorption occurs along an entirety of the absorbent 34 without gaps created by non-porous structure(s), such as the above-referenced sleeves and/or shutter devices. As the aspirate contacts the absorbent 34, a portion of the aspirate including aqueous fluids, for example, blood, anesthetic, saline, and oil is wicked through the filter and out of the aspirate, and absorbed into the absorbent.

In the described embodiment, the filter 20 and the second portion 18 of the barrel 14 generally define the absorptive chamber 32. A cap 36 and a seal 38 (as shown in FIG. 2) are supported at a distal end of the barrel 14 preventing leaking along the plunger 26 and further defining the chamber 32. The chamber 32, within which the absorbent 34 is positioned, is in fluid communication with the second channel along at least a length of the filter 20. As shown in FIG. 1, the chamber 32 is defined and extends along substantially an entire length of the second portion 18 of the barrel 14.

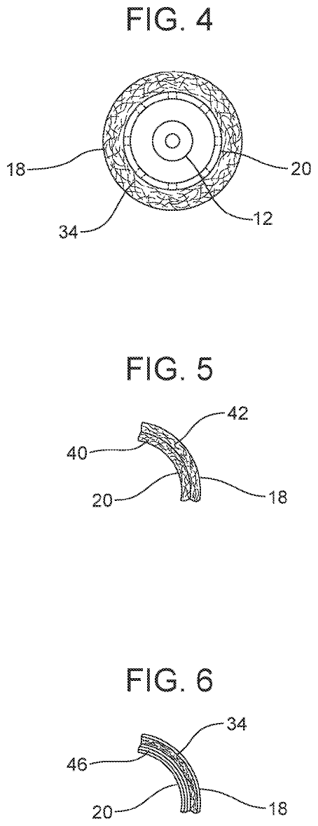

The absorbent 34 may simply be any material capable of retaining fluid. For instance, the absorbent 34 may include a cotton, a super absorbent-embedded cotton, a superabsorbent material, or a superabsorbent polymer. In the described embodiment, however, the absorbent 34 is a multi-component absorbent arranged in a homogenous manner as shown in FIG. 4.

The multi-component absorbent 34 includes fibers which wick and/or absorb oils from ruptured fat cells. Such fibers may include, for example, combed or straightened cotton fibers that can soak up oil by letting it flow into channel-like spaces that form between the fibers relying on capillary action. Further, cotton fibers provide both adsorption wherein the oil merely adheres to the fibers and absorption into the fibers. A second fiber component includes fibers which wick and/or absorb aqueous fluids such as saline, blood, and local anesthetic and a third component includes a material for trapping or locking in the wicked fluids. Such a material may include, for example, a sodium polyacrylate or other superabsorbent material as is known in the art. All three of these components, or combinations of any two or more, may be arranged in such a way as to maximize their effectiveness.

In another embodiment, the previously mentioned absorbent components may be situated in layers arranged and varied in either a vertical, a radial, or an angular pattern with respect to a central axis of the syringe 10. As shown in FIG. 5, a first layer 40 may include the first and second fiber components for wicking oils and aqueous fluids, and a second layer 42 may include the material for locking in the wicked oil and fluid.

As shown in FIG. 6, the absorbent may be configured as a cartridge 44 in another embodiment and inserted into the chamber 32 as a unit during manufacture or at the time of use. The cartridge 44 may be stamped or otherwise formed and slid over the filter 20 and into the chamber. In this embodiment, a filter 46 is utilized to prevent viable fat cells from passing out of the channel into the chamber 32. The filter 46 extends within the chamber 32 and is positioned between the filter 20 and the absorbent 34. While not limited to these examples, the filter 46 may be a cotton gauze, a mesh filter, a fine mesh filter, a paper, an absorptive paper, a semipermeable membrane, and/or a perforated polymer sheet. It should be noted that a filter, similar to filter 46, may be utilized in non-cartridge embodiments as well to preclude fat cells from passing through the filter 20 into the absorbent chamber 32. Even more, a color indicator may be incorporated into any of the described embodiments to indicate to a user when a sufficient amount of fluid has been absorbed. Such an indicator would be visible through at least a transparent or semi-transparent portion of the syringe barrel 14.

Returning to FIG. 3, the filter 20 forms a portion of the filter assembly 30. A first or proximal support 48 receives a first end of the filter 20. As best shown in FIG. 2, the proximal support 48 is generally annular in shape. A notch 50 defined by the barrel 14 is shaped to receive the proximal support 48. Once engaged, longitudinal movement of the filter 20 within the barrel 14 and lateral movement of its proximal end are precluded. In addition, aspirate drawn into the first channel is precluded from entering the absorptive chamber 32 except through the filter 20. To accomplish this sealing effect, the proximal support 48 may be made from a suitable material (e.g., a hard rubber) or a gasket or O-ring (not shown) may be added as is known in the art.

As further shown in FIGS. 2 and 3, a second support 52, similarly generally annular in shape, receives a second end of the filter 20. A support surface 54 extends radially and engages the second portion 18 of the barrel 14 limiting lateral movement of the filter therein. A circular ring 56 extends longitudinally from the support surface 54 forming a ledge 58. The ledge 58 supports the gasket or O-ring 38 within the circular ring 56. In the described embodiment, the second support 52 engages the cap 36 trapping the sealing gasket 38 in position therebetween for preventing leakage along the plunger 26.

The first and second supports 48 and 52 also hold the filter 20 mesh in its cylindrical configuration. Further, a small flange (not shown) located distally on the first support 48 and proximally on the second support 52 engages and protects raw ends of the filter 20 and provides a stop for assembly and a smooth transition as the plunger gasket 28 moves from the inner wall 24 of the barrel first portion 16 to inside of the filter.

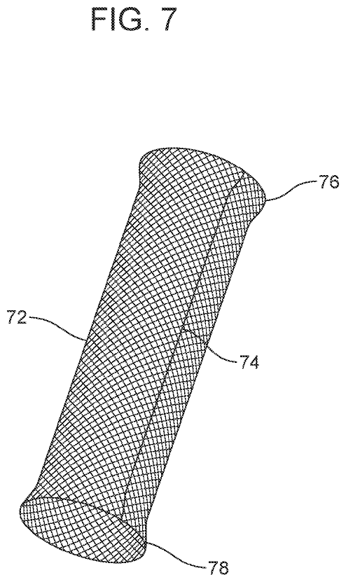

In an alternate embodiment shown in FIG. 7, a filter 72 is formed into a cylindrical shape using a laser weld 74 or similar, known technique. In this manner, the first and second supports 48 and 52 are no longer required to maintain the cylindrical shape. In addition, a proximal end 76 and a distal end 78 of the filter 72 are flared outward to provide a smooth transition for the plunger gasket 28. The flared ends 76 and 78 fit into the same recesses in the barrel 14 used to accommodate filter 20.

More specifically, proximal end 76 may be received in notch 50 defined by the barrel 14. Once engaged, longitudinal movement of the filter 72 within the barrel 14 and lateral movement of its proximal end 76 are precluded. In addition, aspirate drawn into the first channel is precluded from entering the absorptive chamber 32 except through the filter 72. To accomplish this sealing effect, a gasket or O-ring (not shown) may be added as is known in the art. Similarly, distal end 78 engages the second portion 18 of the barrel 14 limiting lateral movement of the filter 72 therein. The cap 36 may be altered in such an embodiment to accommodate a gasket or O-ring for preventing leakage along the plunger 26 as is known in the art.

As described above and in all embodiments, retraction of the plunger 26 from a first position draws the aspirate from the harvest site of the body into the barrel 14 of the syringe 10. Retraction beyond the second position exposes the aspirate to the absorbent 34 for absorbing a first portion of the aspirate while leaving a second portion of the aspirate, including primarily viable fat cells, in the channel of the syringe 10.

In the described embodiment, a detent 52 formed in the plunger 26 engages the seal 38 to provide a noticeable change in plunger movement to alert a user that the plunger has been withdrawn a certain distance. In this instance, the detent 52 is formed such that the alert occurs when the plunger 26 is withdrawn a sufficient distance to expose approximately two-thirds of the absorbent chamber 32 to the aspirate. At this position, primary absorption of the aspirate can occur allowing the user to simply agitate the aspirate and/or to subsequently draw a saline wash into the syringe 10 as is described in more detail below. Such a two part absorptive process yields an overall higher quality fat graft. Of course, more than one detent may be utilized in an alternate embodiment in association with absorptive processes having three or more absorptive steps.

In accordance with a method of securing fat cells for micro fat grafting, the plunger 26 is returned at least to the second position such that the second portion of the aspirate is no longer exposed to the absorbent 34. In this position, the second portion of the aspirate including the viable fat cells is positioned within the first portion 16 of the barrel 14 and may be returned to a grafting site of the body.

As described above, different embodiments of the syringe 10 include varying absorbents and include a filter 20 to aid in separating the liquid portions of the aspirate from the viable fat cells. In such embodiments, the exposing step may further include wicking the first portion of the aspirate and/or filtering the aspirate. Even more, with the plunger 26 retracted from the first position such that the aspirate is exposed to the absorbent 34, the syringe 10 may be agitated to promote absorption. Agitating the syringe 10 may include rolling and/or shaking the syringe by mechanical or physical means and/or rotating the syringe along its long axis. More specifically, an external device may be utilized to roll and/or shake the syringe 10 and aspirate therein or the syringe could be placed on a rotating external appliance which would utilize centrifugal force to drive the fluid portion of the aspirate into the absorbent 34.

Once the viable fat cells are separated from the first or liquid portion of the aspirate and positioned within the barrel 14 of the syringe 10 as described above, the viable fat cells or fat graft is ready for re-introduction to the body. The same syringe 10 may be used to re-inject the viable fat cells or, if the user prefers, the fat graft may be transferred or expelled in one or more portions to one or more smaller syringes. Such transfers to smaller syringes may be accomplished in a closed system utilizing the Luer-Lok connection fitting of the syringe. For instance, a tube may be connected at a first end to the fitting and at a second end to a first smaller syringe, and possibly others.

In using the syringe embodiments described, a puncture incision is made by the user in the desired harvest site of the human body. A harvesting device, such as a cannula, is connected to a syringe by means of a Luer lock fitting, or other fitting, located at the proximal aspect of the syringe barrel. The cannula is inserted into the body; the user withdraws a plunger of the syringe and moves the cannula in a manner familiar to those skilled in the art. A vacuum pressure created in a barrel of the syringe results in aspirate being drawn into the barrel. This vacuum pressure may be created using the plunger and/or a secondary vacuum source. As the procedure continues, the user further withdraws the plunger to maintain a consistent, low vacuum. Graduations printed on the syringe barrel, indentions or raised rings may be utilized to reveal an amount of aspirate collected. In one embodiment, a releasable plunger holding mechanism locks the plunger at whatever position it is withdrawn to, thereby avoiding the need for the user to hold the plunger in the desired position. This process continues until a desired quantity of aspirate has been obtained or at least a proximal aspect of the barrel is full.

The cannula is subsequently removed from the body and the syringe is oriented with a proximal tip pointing up. The surgeon then retracts the plunger fully until it contacts a stop created by a top cap in the described embodiment. In this way, the entire contents of the aspirate are exposed to an absorbent via the filter. Once the plunger is retracted, the syringe can either be left in the vertical position, or lightly agitated in either a rolling, rotating, and/or shaking manner. After a period allowing for fluid absorption, with the syringe held upright, the user returns the plunger thereby pushing the remaining viable fat cells to the proximal aspect of the barrel and expelling any remaining air. The same cannula used for harvesting, a different one, a hypodermic needle, or other re-insertion device is then re-attached to the fitting. The re-insertion device is inserted into the body at a graft site. The plunger is then pushed forward expelling the processed fat graft through the cannula and into the graft site. In an alternate embodiment, the processed graft may be pushed out into another container, or syringe(s), in an open or a closed transfer process. The described syringes are intended for discard after a single use.

In another method, an aspirate may be drawn into the first channel of the first portion 16 of the syringe barrel 14 by suction created by withdrawal of the plunger 26. A wash solution (e.g., saline or lactated ringers solution) in a volume of approximately 25% of the aspirate is similarly drawn into the first channel and added to and mixed with the aspirate. The aspirate/wash mixture is subsequently drawn into the second channel defined by the filter 20 until an indicia is reached in the described embodiment. In this instance, the indicia is detent 52 engaging the seal 38. The mixture is allowed to dwell in this position for a period of time, preferably about one minute, allowing portions of the mixture to traverse the filter and be absorbed by the absorbent 34. Mechanical agitation or rotation of the syringe 10 may be used during this period to provide additional mixing and to ensure maximal exposure to the absorbent 34.

A remaining or non-absorbed portion of the aspirate/wash mixture remaining in the second channel is then returned to the first channel and a second specified quantity (e.g., again about 25% of the initial aspirate) of a wash solution is added using the plunger 26. The subsequent mixture of once processed aspirate and additional wash solution is then drawn into the second channel. This time, however, the syringe 26 is retracted beyond the above-noted indicia allowing an entirety of the filter 20 and absorbent 34 to be exposed to the subsequent mixture. Again, the mixture is allowed to remain in this portion for a period of time, preferably about one minute, allowing portions of the subsequent mixture to traverse the filter and be absorbed by the absorbent 34. Again, mechanical agitation or rotation of the syringe 10 may be used during this second period to provide additional mixing and to ensure maximal exposure to the absorbent 34. This multi-step method results in absorption of a high percentage of undesired free oil(s) and aqueous components of the aspirate mixture. Following the second dwell time, a remaining or non-absorbed portion of the mixture consisting primarily of highly concentrated fat cells is then returned from the second channel to the first channel ready for re-insertion into a graft site as generally described above.

In summary, numerous benefits result from a syringe for withdrawing an aspirate from a body for micro fat grafting and related methods of securing fat cells for micro fat grafting using a syringe. The foregoing has been presented for purposes of illustration and description. It is not intended to be exhaustive or to limit the embodiments to the precise form disclosed. Obvious modifications and variations are possible in light of the above teachings. For example, a plunger locking mechanism may be utilized with any of the described embodiments including, for example, a releasable holding system that locks the plunger in a partially withdrawn position when retracted, a friction lock mechanism, a flexible rubber flange that wedges against the plunger cylinder, an offset collar that wedges against the plunger cylinder, or a ratchet system which indexes against the plunger cylinder at specified points. Each of these locking mechanisms are generally known in the industry and each serves to maintain a vacuum in at least a proximal portion of the syringe barrel. All such modifications and variations are within the scope of the appended claims when interpreted in accordance with the breadth to which they are fairly, legally and equitably entitled.

1-60. (canceled)

61. A syringe for withdrawing an aspirate including fat cells from a body for micro fat grafting, comprising:
a barrel having first and second portions, the first portion having an inner wall with a lesser diameter than an inner wall of the second portion;
a filter for separating fat cells from the aspirate extends within the second portion of the barrel;
an absorbent positioned between the filter and the second portion inner wall for absorbing the remaining aspirate passing through the filter; and
a plunger for longitudinal movement within the barrel along the first portion inner wall and the filter, the plunger having a gasket attached to a proximal end of the plunger forming a seal with the first portion inner wall in a first position and an inner wall of the filter in a second position.

62. The syringe for withdrawing an aspirate including fat cells from a body of claim 61, wherein the barrel second portion inner wall defines a notch for receiving a proximal end of the filter.

63. The syringe for withdrawing an aspirate including fat cells from a body of claim 62, wherein a proximal end of the filter is outwardly flared.

64. The syringe for withdrawing an aspirate including fat cells from a body of claim 62, further comprising a proximal support that receives the proximal end of the filter.

65. The syringe for withdrawing an aspirate including fat cells from a body of claim 64, wherein the proximal support sealingly engages at least the notch.

66. The syringe for withdrawing an aspirate including fat cells from a body of claim 61, further comprising a distal support that receives a distal end of the filter and defines a central aperture through which the plunger extends.

67. The syringe for withdrawing an aspirate including fat cells from a body of claim 66, wherein the distal support includes a support surface extending radially for engaging the second portion of the barrel.

68. The syringe for withdrawing an aspirate including fat cells from a body of claim 66, wherein the distal support further includes an annular ring extending distally from the disk.

69. The syringe for withdrawing an aspirate including fat cells from a body of claim 68, wherein an inner diameter of the annular ring is larger than the diameter of the central aperture forming a distal facing ledge for supporting a third gasket.

70. The syringe for withdrawing an aspirate including fat cells from a body of claim 61, wherein an outer wall of the filter and the barrel second portion inner wall at least partially define a chamber wherein the absorbent resides.

71. The syringe for withdrawing an aspirate including fat cells from a body of claim 70, further comprising a cap engaging a distal end of the barrel and further defining the chamber wherein the absorbent resides.

72. The syringe for withdrawing an aspirate including fat cells from a body of claim 61, wherein the filter is a mesh.

73. The syringe for withdrawing an aspirate including fat cells from a body of claim 61, further comprising a second filter extending within the chamber and positioned between an outer wall of the filter and the absorbent.

74. The syringe for withdrawing an aspirate including fat cells from a body of claim 73, wherein the second filter and the absorbent form a cartridge.

75. The syringe for withdrawing an aspirate including fat cells from a body of claim 61, further comprising at least one detent formed in the plunger for engaging a seal supported by the barrel to provide a tactile change in the plunger movement.

76. A syringe for withdrawing an aspirate including fat cells from a body for micro fat grafting, comprising:
a barrel having first and second portions, the first portion having an inner wall with a lesser diameter than an inner wall of the second portion;
a filter extends within the second portion of the barrel;
an absorbent positioned between the filter and the second portion inner wall for absorbing the remaining aspirate passing through the filter; and
a plunger for longitudinal movement within the barrel along the first portion inner wall and the filter, wherein the filter separates fat cells from the aspirate while allowing the remaining aspirate to traverse the filter.

77. The syringe for withdrawing an aspirate including fat cells from a body of claim 76, wherein the filter is a mesh screen.

78. The syringe for withdrawing an aspirate including fat cells from a body of claim 77, wherein the mesh screen is a plastic polymer or a metal.

79. The syringe for withdrawing an aspirate including fat cells from a body of claim 76, wherein a plurality of fenestrations in the filter are substantially evenly distributed along an entirety of the filter.

80. A method of obtaining fat cells from a body for micro fat grafting using a syringe, comprising the steps of:
retracting a plunger having a gasket attached to a proximal end thereof from a first position wherein the gasket engages an inner wall of a first portion of a barrel of the syringe in order to draw an aspirate including fat cells from a harvest site of the body into the barrel first portion;
further retracting the plunger to a second position exposing the aspirate to a filter for separating fat cells from the aspirate and an absorbent for absorbing a non-fat cells portion of the aspirate, wherein the gasket engages an inner wall of the filter in the second position; and
moving the plunger toward the first position such that an unabsorbed portion of the aspirate including the separated fat cells is no longer exposed to the absorbent.

81. The method of obtaining fat cells from a body for micro fat grafting using a syringe of claim 80, wherein the inner wall of the first portion of the barrel has a lesser inner diameter than an inner diameter of a second portion of the barrel.

82. The method of obtaining fat cells from a body for micro fat grafting using a syringe of claim 80, wherein the inner wall of the first portion of the barrel has an inner diameter substantially the same as an inner diameter of an inner wall of the filter.

83. The method of obtaining fat cells from a body for micro fat grafting using a syringe of claim 80, wherein the moving step includes the step of returning the unabsorbed portion of the aspirate including the separated fat cells to a grafting site of the body.

84. The method of obtaining fat cells from a body for micro fat grafting using a syringe of claim 80, further comprising the step of adding a wash solution to the aspirate drawn in the retracting step by further retracting the plunger from the first position to a first intermediate position.

85. The method of obtaining fat cells from a body for micro fat grafting using a syringe of claim 84, further comprising the step of adding a wash solution to the unabsorbed portion of the aspirate by again retracting the plunger from the first intermediate position to a second intermediate position.

86. The method of obtaining fat cells from a body for micro fat grafting using a syringe of claim 85, further comprising the step of further retracting the plunger along the filter to the second position exposing the unabsorbed portion of the aspirate and wash solution mix to the filter and the absorbent.

87. The method of obtaining fat cells from a body for micro fat grafting using a syringe of claim 80, wherein the step of moving the plunger toward the first position occurs after at least one minute following retraction of the plunger to the second position.
